(19) United States
(12) Patent Application Publication
(10) Pub. No.: US 2005/0278003 A1
(43) Pub. Date: Dec. 15, 2005

(54) Electroluminescent light therapy devices

(76) Inventor: Harold Feldman, Lawrence, NY (US)

Correspondence Address: ABELMAN, FRAYNE & SCHWAB, 666 THIRD AVENUE, 10TH FLOOR, NEW YORK, NY, 10017, US

(21) Appl. No.: 11/128,856

(22) Filed: May 12, 2005

Related U.S. Application Data
(60) Provisional application No. 60/570,943, filed on May 12, 2004.

(51) Int. Cl. A61N 1/00

(52) U.S. Cl. 607/88

(57) ABSTRACT

Wearable devices including a hat or cap, eyeglasses, an eye mask, or a face mask deliver light from light-emitting diodes or deliver electroluminescent light from a light source including an electroluminescent wire or flat panel powered by an inverter connected to the hat or cap, the eyeglasses, the eye mask, or the face mask. The delivery of the light is for entertainment or therapeutic purposes. Delivery of therapeutic light to the retinal area or to the skin of the user by a wearable device, without the use of the hands of the user, applies consistent levels of the therapeutic light to relax the user, to remove wrinkles in the skin, and to impart other healing effects. A sufficient amount of lux at appropriate optical wavelengths is generated to be incident for long periods upon the retina of the user or upon the skin of the user for healing effect.

### CROSS-REFERENCE TO RELATED APPLICATIONS

This application claims priority to U.S. provisional patent application No. 60/570,943, filed May 12, 2004, now abandoned, which is incorporated herein by reference in its entirety.

### BACKGROUND OF THE INVENTION

1. Field of the Invention

This invention relates to light therapy, and in particular to a device which uses electroluminescent light for light therapy.

2. Description of the Related Art

Short wavelengths of light in the blue portion of the electromagnetic spectrum, at wavelengths in the range of about 460 nanometers (nm.) to about 480 nanometers at illumination levels in the range of about 2,000 lux to about 30,000 lux, are most effective at affecting the circadian system of a person. A lux is a measure of illumination equal to one lumen per square meter. The color produced by an electroluminescent (EL) light device emitting such wavelengths simulates a clear blue sky. The EL light device may be powered by an inverter operating at about 2,000 Hz to about 8,000 Hz and about 90 V to about 130 V to produce the proper blue light.

The amount of light produced by an EL wire, EL flat panel, light emitting diodes (LEDs), or a combination of LEDs and EL devices positioned close to the eyes affects the circadian rhythms and may have a beneficial effect on Seasonal Affective Disorders (SADs), Delayed Sleep Phase Syndrome (DSPS), depression, fatigue, jet lag, as well as other maladies. The blue light has an effect on the inhibition, release, and/or excretions of hormones; for example, the blue light inhibits melatonin release or changes the mood-altering neurotransmitters called serotonin. In the prior art, most light therapies employ a light box using an extremely bright full spectrum of light, which a person sits in front of or within for a certain period of time. Such light boxes are not portable, so the user cannot receive such light therapy unless the user is stationary.

In addition, the use of light boxes to provide a bright full spectrum of light, such as white light, is painful to the eyes and therefore makes users uncomfortable, and even temporarily blinded. Therefore, the duration of use must be relatively short, limiting their therapeutic value.

Furthermore, electroluminescent (EL) light sources are not as painful to the eyes, but the delivering the proper amount of lux with known light sources such as EL devices is difficult due to the rapid degradation of inverters used in EL devices of the prior art, and thus the delivery of light to the eyes by EL light sources decreases rapidly. Accordingly, prior art light therapy devices have short operating lifetimes and so are of limited therapeutic value.

Illumination from light sources may also be used for relaxation, for acne treatment, for wrinkle removal, and other known applications; for example, as described in U.S. Pat. No. 6,887,260; and in U.S. patent application publication No. US 2005/0073839 A1, each of which is incorporated herein by reference in its respective entirety. However, the delivery of such illumination is difficult if such light sources are not portable or wearable, such as a light box or a tanning bed which cannot be readily worn or carried about by the user. In addition, portable devices which are carried require the use of one's hands to continually apply the illumination, thus limiting one's free hands for other activities. Furthermore, the application of illumination by hand may promote uneven therapy to the user, since one's hand carrying the therapeutic illumination device may not be held in a constant position as one walks or performs other activities, and the user may tire of holding the carried therapeutic illumination device, which may cause the user to cease the therapy.

### BRIEF SUMMARY OF THE INVENTION

Wearable devices including a hat or cap, eyeglasses, an eye mask, or a face mask deliver light from light-emitting diodes or deliver electroluminescent light from a light source including an electroluminescent wire or flat panel powered by an inverter connected to the hat or cap, the eyeglasses, the eye mask, or the face mask. The delivery of the light is for entertainment or therapeutic purposes. Delivery of therapeutic light to the retinal area or to the skin of the user by a wearable device, without the use of the hands of the user, applies consistent levels of the therapeutic light to relax the user, to remove wrinkles in the skin, and to impart other healing effects. A sufficient amount of lux at appropriate optical wavelengths is generated to be incident for long periods upon the retina of the user or upon the skin of the user for healing effect.

A wearable illumination device is disclosed having a wearable member to be worn by a user; and a light source mounted to the wearable member for emitting light onto a portion of the body of the user for a purpose selected from the group consisting of illuminating the portion of the body for entertainment effects and illuminating the portion of the body for therapeutic effects. The light source is selected from the group consisting of a light emitting diode, an electroluminescent wire, and an electroluminescent panel.

A therapeutic device applies therapeutic light to a portion of the face of the user, with the therapeutic device having a light source for emitting the therapeutic light; and a wearable member worn by the user and having the light source mounted thereon for positioning the light source substantially adjacent to the portion of the face of the user, thereby causing healing effects in the portion of the face. The portion of the face may be the retina of an eye of the user, or may be a portion of the skin of the face of the user, such as an area of skin substantially surrounding the eyes of the user, or substantially the entire face of the user.

The light source may be a light emitting diode, or an electroluminescent device, such as an electroluminescent wire or an electroluminescent panel. A power source may be included which is electrically connectable to the light source for providing electrical power to the light source, thereby causing the emission of the therapeutic light.

The wearable member may be a cap with a brim having the light source mounted on an underside of the brim; or a set of eyeglasses having the light source mounted on the frame of the eyeglasses facing the face of the user; or may be a set of eyeglasses having the light source mounted within a transparent frame of the eyeglasses facing the face of the user; or may be an eye mask substantially covering the skin surrounding the eyes of the user and having the light source mounted on a side of the eye mask facing the face of the user, with an optional light blocking panel for blocking the therapeutic light from entering an eye of the user; or may be a face mask substantially covering the face of the user and having the light source mounted on a side of the face mask facing the face of the user, with an optional a light blocking panel for blocking the therapeutic light from entering an eye of the user.

A kit for light therapy may also be included, with the kit having a wearable device wearable by a user and having a wearable member and a light source for emitting therapeutic light, with the light source mounted on the wearable member for positioning the light source substantially adjacent to the portion of the face of the user, thereby causing healing effects in the portion of the face; and a power source wearable by a user and electrically connectable to the light source for providing electrical power to the light source, thereby causing the emission of the therapeutic light.

The wearable device may be selected from the group consisting of: a cap, eyeglasses, an eye mask, and a face mask; and the light source may be selected from the group consisting of: a light emitting diode, an electroluminescent wire, and an electroluminescent panel.

### BRIEF DESCRIPTION OF THE SEVERAL VIEWS OF THE DRAWINGS

Preferred embodiments of the invention are disclosed hereinbelow with reference to the drawings, wherein:

FIG. 1 is a bottom rear left side perspective view of a cap having a light source;

FIG. 2 is a bottom rear right side perspective view of a set of eyeglasses having a light source;

FIG. 3 is a bottom rear left side perspective view of an eye mask having a light source;

FIG. 4 is a bottom rear left side perspective view of a face mask having a light source;

FIG. 5 is a top front left side perspective view of a user wearing a cap as in FIG. 1;

FIG. 6 is a top front left side perspective view of a user wearing a set of eyeglasses as in FIG. 2;

FIG. 7 is a top front left side perspective view of a user wearing an eye mask as in FIG. 3 with the eye mask having eye holes;

FIG. 8 is a top front left side perspective view of a user wearing an eye mask as in FIG. 3 with the eye mask lacking eye holes;

FIG. 9 is a top front left side perspective view of a user wearing a face mask as in FIG. 4 with the face mask having eye holes; and

FIG. 10 is a top front left side perspective view of a user wearing a face mask as in FIG. 4 with the face mask lacking eye holes.

### DETAILED DESCRIPTION OF THE INVENTION

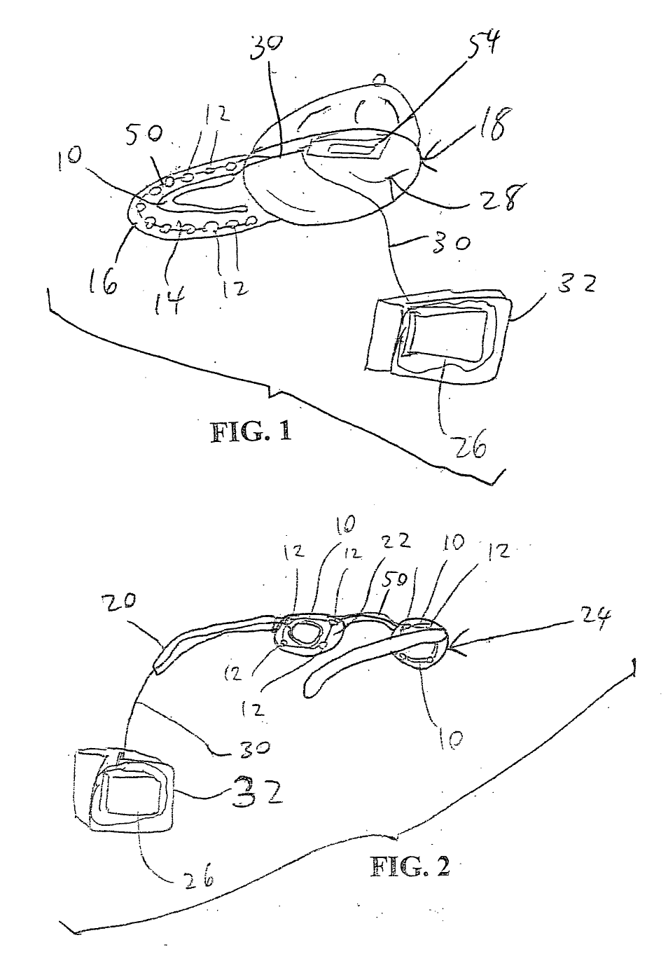

Referring to FIGS. 1-10, light therapy devices in the form of wearable accessories deliver substantially blue light from light sources via electroluminescent (EL) lighting devices 10, such as an EL wire or an EL panel, which emit about 2,000 lux; via substantially blue LEDs 12, for example, up to about twelve LEDs arranged and spaced apart on the wearable accessory to direct the blue light towards a portion of the face of the user, with such a portion of the face including the eyes of the user; the retinas of the eyes of the user; the skin of the face of the user; select portions of the skin of the face such as around the eyes, nose, and mouth of the user; the forehead or brow of the user; or the entire face of the user. It is to be understood that the face of the user includes any exposed or exposable portion of the face or head of the user, including the retinas.

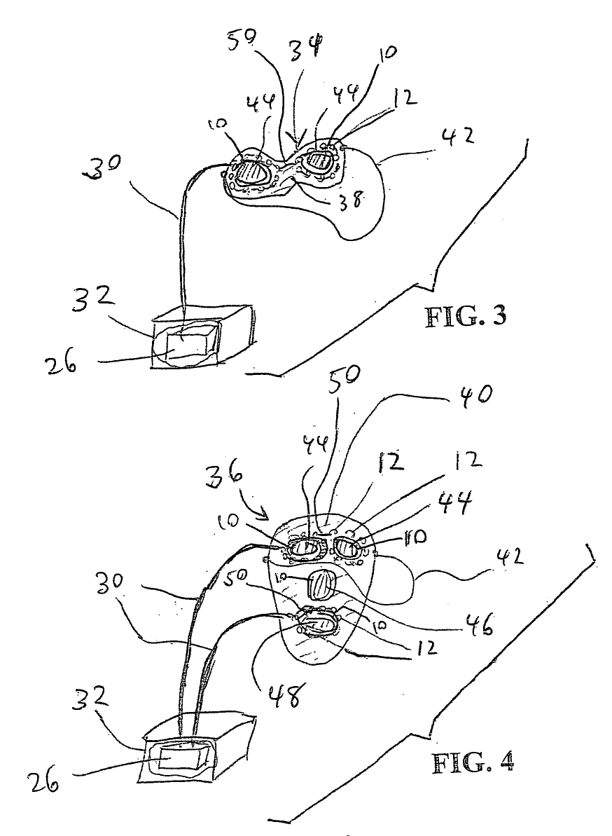

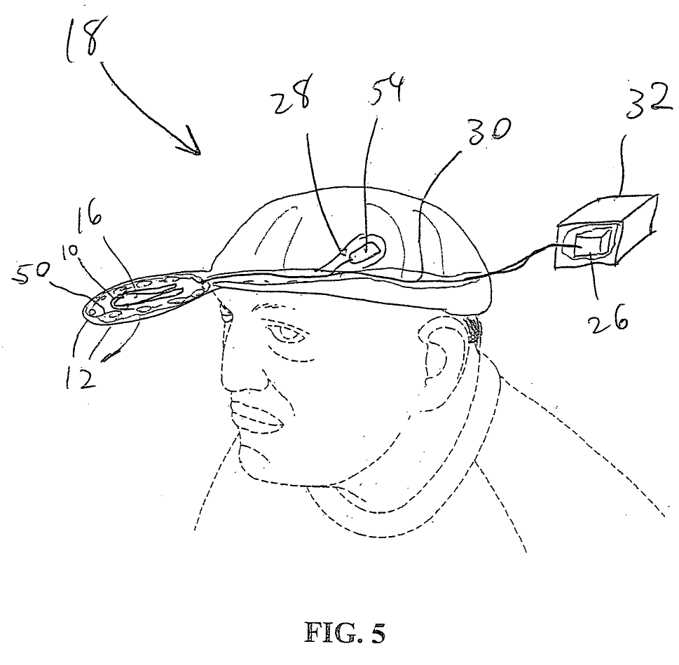

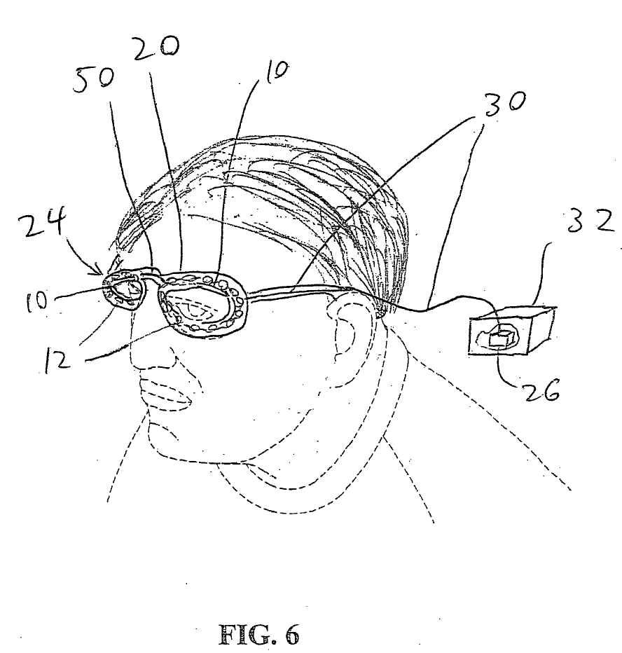

Such light therapy devices have the blue LEDs capable of emitting about 30,000 lux; and/or via a combination of LEDs and EL devices which are sewn, adhered to, and/or otherwise attached to the underside 14 of a brim 16 of a hat or cap 18 such as a baseball cap, as in FIGS. 1 and 5; or under the brim of a sun visor; or on a face shield; or on or within the frames 20 or lenses 22 of eyeglasses 24, as in FIGS. 2 and 6, such as transparent frames allowing light from the light source to pass through to the portion of the face of the user for light therapy; or on masks worn over a portion the face such as eye masks as shown in FIGS. 3 and 7-8; or face masks as shown in FIGS. 4 and 9-10.

If less lux is emitted, more time is needed for the user to be exposed to or experience the blue light to attain the therapeutic effects. All of the above brims 16 of the wearable accessories, and/or the entire pair of eyeglasses 24, and/or the masks are capable of being raised or lowered in order to adjust the position of the light source substantially adjacent to the area of the user to receive such light, such as the retinas or the skin of the user, or to temporarily discontinue use and the application of the light therapy by positioning the light source away from the area of the user to receive such light, such as by positioning the cap, eyeglasses, eye mask, or face mask on top of the head of the user or off of the body of the user.

The light sources such as the EL light source 10 and the LEDs 12 are connected in an electrical circuit in series and/or in parallel using connecting wires 50, with the electrical circuit formed by the EL light source 10, the LEDs 12, and the connecting wires 50 being electrically connected to and powered by a power source 26, such as a portable and/or replaceable battery, which may be rechargeable and which may be placed in the wearable accessory, such as in the interior 28 of the cap 18, or with a lead wire 30 which comes out of the cap 18 or out of the frames 20 of the eyeglasses 24, with the lead wire 30 entering a housing 32 which contains the battery 26, with the housing 32 capable of being clipped onto or inserted into the clothing of the user, such as the pants pocket or belt of the user. Such portable and clippable versions of the light therapy devices of the present invention are advantageous since such devices are more comfortable to the wearer/user and provide a longer lasting supply of power. Additional methods deliver even more light by incorporating EL wires or panels as light sources into eyeglasses 24, goggles, sunglasses, monocles, or other accessories worn near or over the eyes of the user.

The advantages of using EL is that such devices emit EL light providing a substantially uniform light which is diffused at the light source, and such EL light is glare-free and is a form of “cold” or “cool” light in which the light does not warm the user and/or the light source does not become substantially warm, and the EL light source may be flexible. As the wearer of the cap 18 looks up under the brim 16, the wearer gets the feeling of being under a blue sky, which lifts the mood of depressed people. In the alternative embodiments using LEDs, the LEDs are substantially unidirectional in directing the light to the eyes of the wearer, and such LEDs are typically bright. Some users may experience discomfort in looking directly into such LEDs; however, the advantages of using LEDs include the relatively high amount of lux produced by the LEDs, to provide a quicker effect on the internal clocks of the body of the users, and so more quickly inhibiting or releasing various hormones for a positive effect on the user.

Alternative embodiments in the form of a mask of lights, such as an eye mask 34, a face mask 36, or other wearable accessories or devices such as the hat 18 and eyeglasses 24 described herein and shown in FIGS. 1-10 may also be used to remove wrinkles from the sides of the eyes and/or any other portions of the face upon which the light from the EL light source 10 and/or the LEDs 12 is incident. Alternating the sequence of illumination of the LEDs or EL light sources may also be used to control the delivery of the incident therapeutic light. In addition, the various devices with such light sources may be used for specific areas of the face with wrinkles. For example, a hat may be used for addressing forehead wrinkles, while the eyeglasses or eye mask may be used for the skin around the eyes, and a full face mask may be employed to treat the entire face.

By using improved inverters to power the EL light source 10, such as the EL devices and inverters described in commonly-assigned pending U.S. non-provisional patent application Ser. No. 10/843,961, filed May 11, 2004 and entitled ELECTROLUMINESCENT DEVICE, which is incorporated herein by reference, the delivery of therapeutic light from the light therapy devices of the present invention is maintained over a longer period of time than in prior art devices, for example, to maintain the delivery of blue light for at least 700 hours of operation at the same lux levels or higher, thus delivering the proper amount of light to the eye area for a more advantageous and beneficial therapeutic effect.

In a first embodiment of the present invention, FIG. 1 is a bottom rear left side perspective view of a cap 18 having a light source composed of an EL light source 10 and LEDs 12 forming an electrical circuit with connecting wires 50 to be connected to lead wires 30 to a power source such as the power source 26 external to the cap 18, which may be a portable and/or removably attachable power pack having the housing 32 with the battery 26 therein; or alternatively a power source 54 internally mounted in the interior 28 of the cap 18.

FIG. 5 is a top front left side perspective view of a user wearing a cap 18 as in FIG. 1, with a cut-away view showing the EL light source 10, the LEDs 12, and the connecting wires 50 on the underside 14 of the brim 16, and the lead wire 30 extend from the electrical circuit formed by the components 10, 12, 50 out to the housing 32 to the battery 26. In use, the cap 18 is worn by the user, and the housing 32 is removably attached, for example, with a clip, to the clothing of the user. Alternatively, the internal power source 54 may be used separate of or in conjunction with the external power source 26, with the internal power source 54 shown in a cut-away view in FIG. 5 in the interior 28 of the cap 18.

In a second embodiment of the present invention, FIG. 2 is a bottom rear right side perspective view of a set of eyeglasses 24 having a light source such as an EL wire 10 and/or LEDs 12. In use, as shown in FIG. 6, which is a top front left side perspective view of a user wearing the set of eyeglasses 24 as in FIG. 2, the frames 20 of the eyeglasses 24 contain or have mounted thereon the EL wires 10 and/or the LEDs 12. In a cut-away view of FIG. 6, the connecting wires 50 are shown extending between the EL wires 10 and/or LEDs 12, for example, over and/or through the bridge of the frames 20, and the lead wire 30 extend from the electrical circuit formed by the components 10, 12, 50 out of or along the ear pieces of the frames 20 to the battery 26. In use, the eyeglasses 24 are worn by the user, and the housing 32 is removably attached, for example, with a clip, to the clothing of the user.

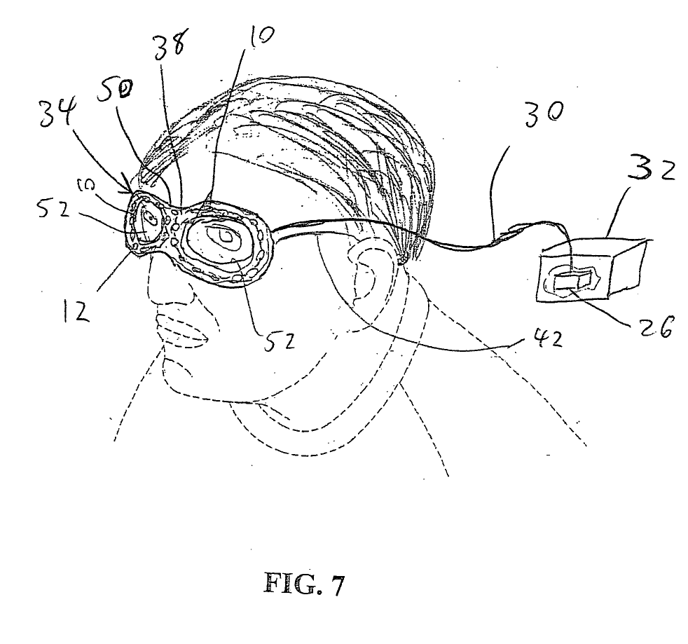

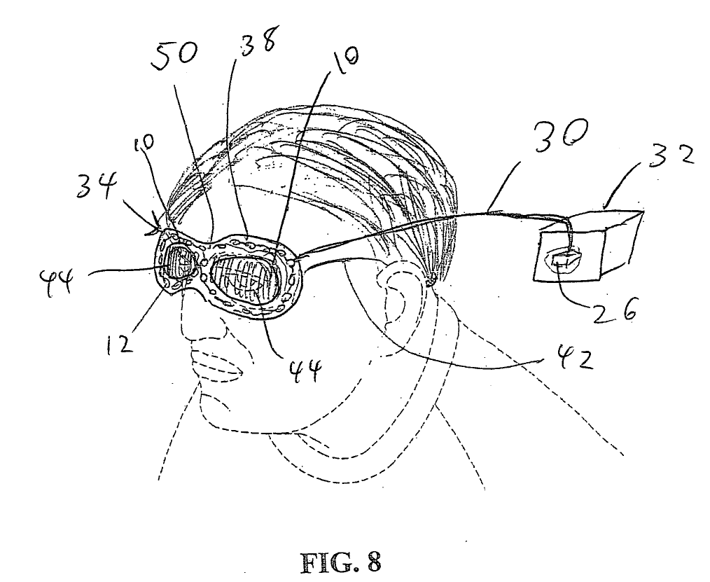

In a third embodiment of the present invention, FIG. 3 is a bottom rear left side perspective view of an eye mask 34 having a light source such as an EL wire 10 and/or LEDs 12 mounted on an eye mask member 38, such as a flexible plastic or cloth piece generally shaped to substantially cover the eyes of the user. In use, as shown in FIGS. 7-8, which are top front left side perspective views of a user wearing the eye mask 34 as in FIG. 3, in a cut-away view, the eye mask member 38 has mounted thereon on the side facing the face of the user the EL wires 10 and/or the LEDs 12. In the cut-away views of FIGS. 7-8, the connecting wires 50 are shown extending between the EL wires 10 and/or LEDs 12, for example, over and/or through the portion of the eye mask member 38 between the eyes of the user, and the lead wire 30 extends from the electrical circuit formed by the components 10, 12, 50 out of the eye mask member 38 to the battery 26. In an alternative embodiment, the lead wire 30 may be connected to or may extend through a portion of the attachment device 42. In use, the eye mask 34 is worn by the user, and the housing 32 is removably attached, for example, with a clip, to the clothing of the user.

The eye mask member 38 is attached to the attachment device 42, such as a string or elastic band, to removably secure the eye mask 34 to the user with the EL light source 10 and/or LEDs positioned substantially adjacent to the eyes of the user for applying therapeutic light to the retinas of the eyes and/or the skin surrounding the eyes of the user.

In one alternative embodiment shown in FIG. 7, the eye mask member 38 includes eye openings 52, allowing the user to see through the eye mask 34, and so the eye mask 34 may be worn anytime during the day, for example, during work, play, or sleep by the user.

In another alternative embodiment shown in FIG. 8, the eye mask member 38 includes eye panels 44 which cover the eyes of the user, preventing the user from seeing through the eye mask 34 and also preventing illumination from the EL light source 10 and/or the LEDs 12 from entering the eyes. Accordingly, the eye mask 34 in FIG. 8 may be used by individuals who are unable to sleep or unable to meditate while awake with such illumination from the EL light source 10 and/or the LEDs 12.

In a further embodiment, the eye panels 44 may include peep holes for allowing the user to see through the eye mask 34 but without the glare from the EL light source 10 and/or the LEDs 12, and so such an embodiment may be used, for example, during driving, reading, or other activities in which the extra light from the EL light source 10 and/or the LEDs 12 may interfere with the activities.

Alternatively, the eye mask 34 in FIG. 7 may be used only during waking hours or active times of day, while the eye mask 34 in FIG. 8 may be used only during sleeping or non-active times of day, such as during meditation.

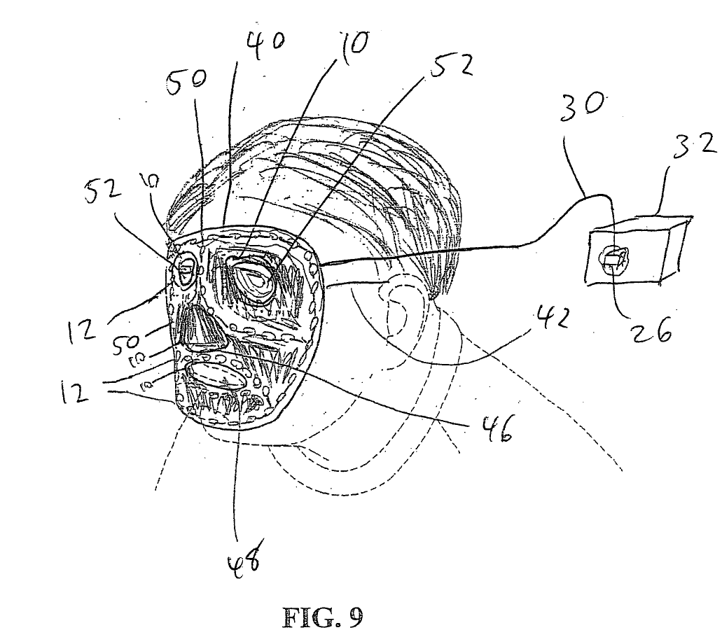

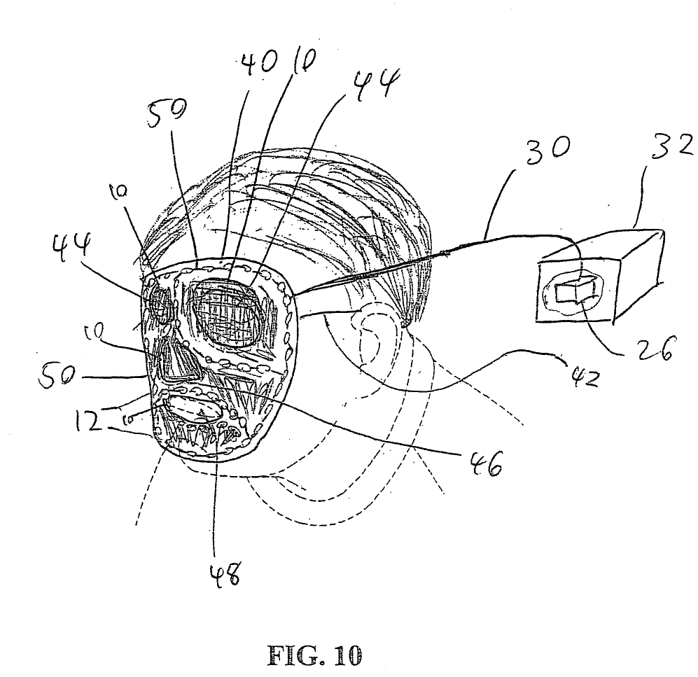

In a fourth embodiment of the present invention, FIG. 4 is a bottom rear left side perspective view of a face mask 36 having a light source such as an EL wire 10 and/or LEDs 12 mounted on a face mask member 40, such as a flexible plastic or cloth piece generally shaped to substantially cover the entire face of the user. In use, as shown in FIGS. 9-10, which are top front left side perspective views of a user wearing the face mask 36 as in FIG. 4, in a cut-away view, the face mask member 40 has mounted thereon on the side facing the face of the user the EL wires 10 and/or the LEDs 12. In the cut-away views of FIGS. 9-10, the connecting wires 50 are shown extending between the EL wires 10 and/or LEDs 12, for example, over and/or through the portion of the face mask member 40 between the eyes of the user as well as around the nose and mouth of the user which are particularly prone to becoming wrinkled or to develop acne or blemishes. It is to be understood that the placement of the EL wires 10 and/or LEDs 12 facing the face of the user may be selectively chosen to apply the therapeutic light to specific portions of the face, such as the forehead, the brow, the cheeks, the chin, and/or other portions of the face which may become wrinkled or may develop acne or blemishes, and so such light therapy is not limited to the regions surrounding the eyes, nose, and mouth of the user.

The lead wire 30 extends from the electrical circuit formed by the components 10, 12, 50 out of the face mask member 40 to the battery 26. In an alternative embodiment, the lead wire 30 may be connected to or may extend through a portion of the attachment device 42. In use, the face mask 36 is worn by the user, and the housing 32 is removably attached, for example, with a clip, to the clothing of the user.

The face mask member 40 is attached to the attachment device 42, such as a string or elastic band, to removably secure the face mask 36 to the user with the EL light source 10 and/or LEDs positioned substantially adjacent to the eyes and skin of the user for applying therapeutic light to the retinas of the eyes and/or the skin of the face of the user.

In one alternative embodiment shown in FIG. 9, the face mask member 40 includes eye openings 52, allowing the user to see through the face mask 36, as well as a nose opening 46 and a mouth opening 48 for normal activities such as breathing, talking, and eating, and so the face mask 36 may be worn anytime during the day, for example, during work, play, or sleep by the user.

In another alternative embodiment shown in FIG. 10, the face mask member 40 includes eye panels 44 which cover the eyes of the user, preventing the user from seeing through the face mask 36 and also preventing illumination from the EL light source 10 and/or the LEDs 12 from entering the eyes. Accordingly, the face mask 36 in FIG. 10 may be used by individuals who are unable to sleep or unable to meditate while awake with such illumination from the EL light source 10 and/or the LEDs 12.

In a further embodiment, the eye panels 44 may include peep holes for allowing the user to see through the face mask 36 but without the glare from the EL light source 10 and/or the LEDs 12, and so such an embodiment may be used, for example, during driving, reading, or other activities in which the extra light from the EL light source 10 and/or the LEDs 12 may interfere with the activities.

Alternatively, the face mask 36 in FIG. 9 may be used only during waking hours or active times of day, while the face mask 36 in FIG. 10 may be used only during sleeping or non-active times of day, such as during meditation.

Each of the various wearable devices 18, 24, 34, 36 may be combined with the power source 26 to form a kit which may be sold together, or the power source 26 may be sold separately but may be interchangeable with compatible plugs of the lead wires 30 of the various wearable devices 18, 24, 34, 36.

In alternative embodiments and uses, the disclosed wearable devices such as hats, caps, eyeglasses, masks, etc., have the ability to be used for entertainment purposes, such as face painting with light. The lights may be, for example, in the colors of the American flag or an athletic team, or may be configured to create a team logo or any design illuminated on the face or any other body part of the wearer. Such illumination effects may be accomplished by placing different colors of LEDs on the cap, visor, or eyeglasses, or by mounting and orienting a flashlight or other light sources onto the wearable devices to cause a display of light on the face of the wearer. For example, one LED may be painted with a logo such that, when the LED is lit, the painted logo on the LED causes the logo to appear in light on the face of the wearer.

Accordingly, the present invention is not be limited to medical or therapeutic uses, but may also be applied to any possible use of the devices described herein. The present invention has general application and encompasses any known or possible use and variation of illumination. For example, the present invention may use any or all colors of LEDs, of EL wires and fibers, or EL flat panels. In addition, the present invention may also include the ability to adjust an LED and optionally to pinpoint certain parts of the face by the light from the LED; for example, with the LED being placed in a ball and socket to allow for maximum movement of the LED.

Furthermore, the uses and applications of the wearable illumination devices of the present invention are not limited to facial areas, since such wearable illumination devices may be designed to fit any body part, any portion thereof in any orientation, and also not limited to humans, but may be used for entertainment, therapeutic, or other effects of illumination of animals, pets, plants, inanimate objects, etc., since the present invention employs broad and unlimited uses of illumination, as described herein.

While the preferred embodiment of the present invention has been shown and described herein, it will be obvious that such embodiment is provided by way of example only. Numerous variations, changes and substitutions will occur to those skilled in the art without departing from the invention herein. Accordingly, it is intended that the invention be limited only by the spirit and scope of the appended claims.

1. A wearable illumination device comprising:
a wearable member to be worn by a user; and
a light source mounted to the wearable member for emitting light onto a portion of the body of the user for a purpose selected from the group consisting of illuminating the portion of the body for entertainment effects and illuminating the portion of the body for therapeutic effects.

2. The wearable illumination device of claim 1, wherein the light source is selected from the group consisting of a light emitting diode, an electroluminescent wire, and an electroluminescent panel.

3. A therapeutic device for applying therapeutic light to a portion of the face of the user, the therapeutic device comprising:
a light source for emitting the therapeutic light; and
a wearable member worn by the user and having the light source mounted thereon for positioning the light source substantially adjacent to the portion of the face of the user, thereby causing healing effects in the portion of the face.

4. The therapeutic device of claim 3, wherein the portion of the face is the retina of an eye of the user.

5. The therapeutic device of claim 3, wherein the portion of the face is a portion of the skin of the face of the user.

6. The therapeutic device of claim 5, wherein the portion of the skin is an area of skin substantially surrounding the eyes of the user.

7. The therapeutic device of claim 5, wherein the portion of the skin is substantially the entire face of the user.

8. The therapeutic device of claim 3, wherein the light source is a light emitting diode.

9. The therapeutic device of claim 3, wherein the light source is an electroluminescent device.

10. The therapeutic device of claim 9, wherein the electroluminescent device is an electroluminescent wire.

11. The therapeutic device of claim 9, wherein the electroluminescent device is an electroluminescent panel.

12. The therapeutic device of claim 3, further comprising:
a power source electrically connectable to the light source for providing electrical power to the light source, thereby causing the emission of the therapeutic light.

13. The therapeutic device of claim 3, wherein the wearable member is a cap with a brim having the light source mounted on an underside of the brim.

14. The therapeutic device of claim 3, wherein the wearable member is a set of eyeglasses having the light source mounted on the frame of the eyeglasses facing the face of the user.

15. The therapeutic device of claim 3, wherein the wearable member is a set of eyeglasses having the light source mounted within a transparent frame of the eyeglasses facing the face of the user.

16. The therapeutic device of claim 3, wherein the wearable member is an eye mask substantially covering the skin surrounding the eyes of the user and having the light source mounted on a side of the eye mask facing the face of the user.

17. The therapeutic device of claim 16, wherein the eye mask includes a light blocking panel for blocking the therapeutic light from entering an eye of the user.

18. The therapeutic device of claim 3, wherein the wearable member is a face mask substantially covering the face of the user and having the light source mounted on a side of the face mask facing the face of the user.

19. The therapeutic device of claim 18, wherein the face mask includes a light blocking panel for blocking the therapeutic light from entering an eye of the user.

20. A kit for light therapy, the kit comprising:
a wearable device wearable by a user and having:
a wearable member; and
a light source for emitting therapeutic light, with the light source mounted on the wearable member for positioning the light source substantially adjacent to the portion of the face of the user, thereby causing healing effects in the portion of the face; and
a power source wearable by a user and electrically connectable to the light source for providing electrical power to the light source, thereby causing the emission of the therapeutic light.
